(19) United States
(12) Patent Application Publication
(10) Pub. No.: US 2015/0282792 A1
(43) Pub. Date: Oct. 8, 2015

(54) ENDOSCOPIC INSTRUMENT

(71) Applicant: Richard Wolf GmbH, Knittlingen (DE)

(72) Inventors: Sören Munnig, Walzbachtal (DE); Frank WEHRHEIM, Bretten (DE)

(21) Appl. No.: 14/679,260

(22) Filed: Apr. 6, 2015

(30) Foreign Application Priority Data
Apr. 7, 2014 (DE) 10 2014 206 653.0

(51) Int. Cl. A61B 17/00

(52) U.S. Cl. CPC A61B 17/00234; A61B 2017/00323; A61B 2017/0034; A61B 2017/00305

(57) ABSTRACT

An endoscopic instrument includes an elongate hollow body and at least one pull element axially movably mounted therein. The pull element in a longitudinal extension is formed from at least two flexible sections. A first section is designed in a mechanically stronger manner than a second section. The second section is designed in a more flexible manner than the first section.

### CROSS REFERENCE TO RELATED APPLICATIONS

This application claims the benefit of priority under 35 U.S.C. §119 of German Patent DE 10 2014 206 653.0 filed Apr. 7, 2014, the entire contents of which are incorporated herein by reference.

### FIELD OF THE INVENTION

The present invention relates to an endoscopic instrument with an elongate hollow body and with at least one pull element which is axially movably mounted therein.

### BACKGROUND OF THE INVENTION

With regard to known endoscopic instruments with movable instrument parts at the distal end, pull elements which engage through the shank of the instrument in an axially movable manner are often formed by rigid rods. Cable systems which are guided in the shank via rollers are also known, for example, in order to realize a pull transmission in an angled or bent instrument, wherein the cables can also be connected to rigid rods in sections.

The pull elements which are formed by rods mostly have quite a high intrinsic weight, so that the weight of the endoscopic instrument is noticeably increased, which renders the handling more difficult. Known cable systems in endoscopic instruments usually have a quite high extensibility over their complete length. This negatively influences the dynamics and closed-loop control performance, with the use of the instrument with a robotic system for instance. The elastically stored energy can moreover endanger the safety of the patient. If the pull cables are designed in a thickener manner, then this leads to a poorer deflection ability around the cable rollers and also to a larger diameter of the instrument.

### SUMMARY OF THE INVENTION

Against this background, it is an object of the invention to form an endoscopic instrument with adequately tensionally strong pull elements which are poorly elastic over their entire length and are simultaneously flexible at least in regions.

The endoscopic instrument according to the invention comprises an elongate (longitudinally extended) hollow body and at least one pull element which is axially movably mounted therein and which is formed of at least two flexible sections in its longitudinal extension, wherein a first section has mechanical characteristics which are different to the second section. Both sections for example can be differently flexible or pliable, i.e. they can each be differently bendable, i.e. elastically bendable, transverse to their longitudinal extension. One of the two sections however can also be designed in a flexural rigid manner. Moreover, the first section for example can have a different extensibility, bending stiffness and/or a generally different mechanically strength in comparison to the second section. Thus, for example, the first section can be formed of a mechanically stronger material and/or have a greater cross section than the second section.

The at least one pull element according to the invention always comprises at least the first as well as the second section. The sequence of the first and second section in the pull element can thereby differ depending on the embodiment. The combination of the first and second section permits the thus formed pull element to have different mechanical characteristics at the first section than at the second section. The arrangement of the at least two sections along the longitudinal extension of the pull element thus depends preferably on the local demands on the pull element. Thus, it can be advantageous to arrange a more flexible section at a certain location of the pull element, and a mechanically more stable section distally thereof; but it can also be advantageous for the pull element to have a mechanically more stable section in a proximal region and a more flexible section in a region which is distally subsequent to this.

Apart from this first and second section, the pull element can comprise further sections. The further sections thereby can have similar or the same characteristics as the first or the second section, but can also differ from the first and second section in their design. These further sections thereby in the longitudinal direction can be formed between as well as in front of or behind the first and second section, in the pull element. The pull element thus by way of the individual sections is designed in a locally flexible or mechanically stable manner, as is necessary with regard to the demands on the endoscopic instrument. If a pull element has a second section distally of a first section, then it can be advantageous if a further mechanically differently designed section then connected for example to a tool connects distally on the second section.

Particularly preferably, the first section has a greater axial stiffness than the second section. The first section is thus less extensible along the longitudinal extension of the hollow body compared to the second section. The extensibility of the pull element as a whole is thus reduced over the complete length. The second section is preferably designed in a more flexible manner than the first section. It can be more pliable than the first section. Thus, for example, the second section can be arranged precisely in a region in the hollow body, in which region deflections for the pull element are located. The second section can then be deflected without any problem on account of its flexibility. With such a deflection for the pull element, it can be the case for example of a deflection in a joint of the endoscopic instrument, wherein the deflection permits the pull element to be led through the joint, even if this is bent. With regard to the deflection element, it is preferably the case of a deflection roller or an arcuate guide. The pull element in such a design can serve for the actuation of the joint itself or however can be led through the joint, in order to move tools or further joints, which are distanced to the joint. An increased flexibility of the pull element with a reduced diameter is necessary when guiding pull elements through joints, in order to be able to ensure an adequately small total diameter of the endoscopic instrument. On account of the design, according to the invention, it is possible to design one pull element or several pull elements in a suitably flexible manner only in the regions, in which such a deflection is actually effected. Other regions which are not led through such joints or deflections in contrast can be designed in a more robust, in particularly tensionally strong manner.

The pull element thus has a combination of the first and second section, so that the thus formed pull element is particularly flexible, i.e. particularly pliable, only at the locations in the context of the second section, where it is necessary due to the design of the endoscopic instrument, for example at deflections in the hollow body, whereas the rest of the pull element is preferably designed in a mechanically robust manner, in particular has a low extensibility.

The first section is preferably arranged at a proximal end of the hollow body, and the second section at a distal end of the hollow body. This means that the first section in the hollow body is situated closer to the proximal end, whereas the second section is situated closer to the distal end. The pull element in the proximal region is designed in a mechanically stable manner, and in the distal region is designed in a flexible manner. The greater flexibility of the pull element at the distal end can be achieved for example by way of a diameter of the second section which is thinner compared to the diameter of the first section, which is why e.g. the hollow body of the endoscopic instrument can also be designed more thinly at its distal end. The diameter of an access in a body, into which the endoscopic instrument is to be inserted, can thus be kept small.

In a particularly preferred design of the invention, at least one of the sections is formed by twisted, stranded and/or braided strands or filaments. The characteristics, in particular the flexibility or mechanical strength of the individual sections, thereby result from the material selection of the strands, from their diameter, from their number and/or from the type of working/processing of the strands, i.e. from the laying of the strands. The sections of the pull element which are formed from strands can be simply adapted to the demands which are placed on it. In particular, a flexible, for example pliable design is rendered possible on account of the strands. As a whole, sections formed by strands have a high tensile strength, so that it is advantageous if the pull element is formed completely from strands. If at least the first and second section is formed by strands, then as previously described, the different mechanical strengths and flexibilities of the sections for example can be set by way of the number of the strands which are present in the section in each case. The strands are preferably manufactured of metal and/or of plastic. Alternatively and/or supplementarily, at least one section of the pull element can be formed of one or more filaments. Filaments can consist of fibers for example. These in particular have no kinking/buckling stiffness, so that the filaments can be deflected on the deflection rollers, without being kinked, which is to say abruptly bent.

Only strands are mentioned hereinafter for the sake of simplicity, but this also includes filaments in the context of the invention.

The first section preferably has a greater cross section than the second section. The greater cross sections permits the mechanically firmer or stronger design of the first section compared to the second section. Thus, for example, both sections can be formed by the same material wherein the greater diameter of the first section leads to the greater mechanical stability. If both sections are formed by strands, the strands e.g. of the first sections can be designed more thickly than the strands of the second section, which as a whole increases the diameter of the first section. Alternatively or additionally, the first section comprises more strands than the second section. Strands can be laid around an axially extending core, given a cable-like design of at least the first section of the pull element. Thus, different cross sections of the first and second section can be achieved by way of different diameters of the individual stands, by way of a different number of the strands laid in a cable-like manner and/or by way of laying strands around a core. A core can extend through both sections in a preferred embodiment.

At least one section comprises a covering on the outer periphery in a preferred embodiment. The covering at least partly encompasses or encloses this section at the outer periphery. Such a covering or encasing of a section, for example in the form of a sleeve, can permit a design of the first section which is mechanically stronger compared to the second section, wherein the first section without the covering can be designed identically to the second section. Alternatively, the first and the second section can be designed differently, i.e. differently in diameter, also within a covering. Alternatively or additionally, such a covering can moreover be designed for example as an outerperipheral, electrical or magnetic insulation of the section.

According to a further preferred embodiment, at least the first and the second section have different electrical and/or magnetic characteristics. The two sections thus can not only differ in their flexibility and mechanically strength, but also differ in their magnetic and/or electrical characteristics.

For HF-instruments for example, the second section is particularly advantageously designed in an electrically insulating manner. This prevents a flow of current through the second section.

Further preferably, the first and the second section are nonpositively, positively and/or materially connected to one another. The connection of the two sections can be designed in a different manner. The first section for example can be bonded to the second section, welded to it and/or connected to it by way of mutual meshing or inter-engagement of the two sections.

In a particularly preferred embodiment, the first section comprises an end which faces an end of the second section, wherein a coupling location connecting these two sections to one another is formed on and/or between the ends of the two sections which face one another. The two sections are arranged adjacently one another, wherein a longitudinal end of the first section is adjacent a longitudinal end of the second section. The ends of the two sections which face one another, in the coupling section can be connected for example by way of soldering, bonding, clamping or in another suitable manner. These two ends which face one another form the coupling location which connects the two sections to one another.

Preferably, the coupling location is formed within a distal half, preferably within a distal third of the hollow body. If for example, only the first and the second section form the pull element, then one of these two sections is designed in such a longitudinally extended manner that it reaches at least from the proximal end to into the distal half of the hollow body, whereas the other section is this formed within the distal half of the hollow body. The coupling location is arranged where the one section ends and the other section begins, i.e. the coupling location connects both sections. If the first section is the proximal, longitudinally extended section, then the pull element in this long section is preferably designed in a mechanically stronger manner. The second section is then arranged in the distal half of the hollow body. The hollow body can be designed in a tapered manner in its distal half, if this second section e.g. has a smaller diameter compared to the first section.

It can be advantageous, depending on the demands placed on the endoscopic instrument, if one section of the two sections extends from the proximal end of the hollow body to into the distal third of the hollow body and thus the other section, connecting to this section extends in the distal third to the distal end of the hollow body. If the first section forms the proximal part of the pull element, then a large part of the pull element is formed by the mechanically stronger first section. Thus, as a whole, the complete stretching of the pull element can be reduced, but an adequate flexibility simultaneously ensured in the distal region. If a deflection is arranged in the distal third for example, then the at least one pull element can follow the defection on account of the flexibility of the second section. Thus, moreover, the distal third of the hollow body can e.g. also have a radially tapered design for example, in the case that this second section is designed more thinly in cross section than the first section.

Particularly preferably, a transition piece peripherally engaging around the ends of the two sections which face one another is arranged at the coupling location. The transition piece is thus arranged on the pull element at the outer periphery and connects the two sections to one another. The transition piece can press radially on the two sections due to the peripheral arrangement of the transition piece, and this leads to a nonpositive fit between the transition piece and the two sections. The transition piece moreover thus encloses the ends of the sections, so that a fraying of the ends can be prevented for example.

The first section in a further preferred embodiment comprises a local thickening (swelling) which is embraced or encompassed by a radially outwardly widened part of the second section. The ends of the two sections which face one another thus not only bear on one another, but they overlap along the longitudinal extension of the pull element. The local thickening is preferably formed on the mechanically more stable first section and the cross section of this section widens with respect to the remaining extension of the section. Further preferably, a transition piece is formed on the local thickening, preferably in a manner facing the first section, and this transition piece at the local thickening embraces the first section as well as the part of the second section which radially embraces the first section, and connects these to one another. The transition piece thereby in particular effects a radial positive fit between the first and the second section. This radial positive fit on account of the widening leads to a positive fit between the two sections along the longitudinal extension. The connection can alternatively be designed the other way round, which is to say the local thickening can also be formed on the second section.

Further preferably, the end of the second section which faces the first section can be radially tapered with respect to the widened part and encompasses the first section behind the thickening. The end of the second section which faces the first section thereby preferably encompasses not only the thickening, but also a part of the first section which is thinner compared to the thickening and which is adjacent the side of the thickening which is away from the second section. A transition piece in the form of an annular clip is thereby advantageously arranged in an outerperipheral manner on the part of the second section which embraces the first section behind the thickening, and effects a non-positive fit with the first section and the second section. The transition piece additionally together with the local thickening of the first section can also effect a positive fit in the longitudinal direction of the pull element. Thus, the transition piece seen in the direction of the longitudinal extension of the pull element can comprise an axial end which bears directly on the local thickening, i.e. the part of the second section which directly surrounds the local widening. The local widening together with the part of the first section and of the second section, said part surrounding the local widening, can be pulled against the transition piece and be held together due to the positive fit, during the pulling on the pull element.

Particularly preferably, at least one strand passes through at least the first and the second section. The first section and the second section thus at the coupling location are not completely separated, but at least one strand is arranged in the first as well as in the second section and extends through both sections. If the first as well as the second section are designed in a stranded manner, then the strand extending through both sections can e.g. form the initially specified core. The continuous strands connect the two sections to one another in an extremely stable manner. The strands forming the individual section lead to the different characteristics or properties of the first section (mechanically strong) and of the second section (flexible), wherein the more flexible second section is preferably formed by a diameter which is smaller compared to the first section. Preferably, the second thinner section extends completely into the first section and is surrounded there by additional strands.

A distal end of the pull element is preferably connected to a tool or a tool part, in a movement-transmitting manner. The design of the pull element with at least one first and a second section permits a low-loss transmission of force and movement, onto the tool. The shank of the endoscopic instrument which is formed by the hollow body can be adapted to the respective tasks of the endoscopic instrument and in particular of the tool, wherein the pull element can be designed by at least the first and second section in a shape which is adapted to the fashioning and function of the endoscopic instrument. The hollow body can be designed e.g. in a bent or arcuate manner in the region, through which the second section extends, on account of an increased flexibility of the second section compared to the first section, wherein the pull element can move through this bending or curvature without any problem due to the flexibility of the second section. The pull element however can simultaneously have a greater mechanical stability in the regions without a bending, on account of the first section.

According to a preferred embodiment, a handle and/or a grip piece or an interface for the attachment on a robot arm is arranged on a proximal end of the instrument. The pull element can be deflected or moved via the handle and/or the grip piece. A tool can be actuated accordingly by the movement in the case that this is arranged at the distal end of the endoscopic instrument.

If an interface for the attachment on a robot arm is arranged on the proximal end, then the pull element can be moved via suitable mechanics on the robot arm. The extension or stretching of the pull element which is reduced on account of the design of the pull element by way of at least one first and second section is indeed advantageous for an endoscopic instrument controlled by robot, since a precise control of a tool arranged distally on the endoscopic instrument is rendered possible via the pull element due to the reduced stretching of this pull element.

The invention is hereinafter explained in more detail by way of embodiment examples which are represented in the drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a perspective view of a first embodiment of an endoscopic instrument according to the invention;

FIG. 2 is an opened, enlarged representation of the detail II in FIG. 1;

FIG. 3a is a perspective view of an embodiment of a coupling location between two sections of a pull element;

FIG. 3b is a perspective view of an embodiment of a coupling location between two sections of a pull element;

FIG. 4 is a perspective view of an alternative embodiment of the coupling location;

FIG. 5 is a perspective view of an alternative embodiment of an endoscopic instrument according to the invention;

FIG. 6 is a perspective view of a further alternative embodiment of the endoscopic instrument; and

FIG. 7 is a perspective view of a robot with a connected endoscopic instrument according to FIG. 6.

### DESCRIPTION OF THE PREFERRED EMBODIMENTS

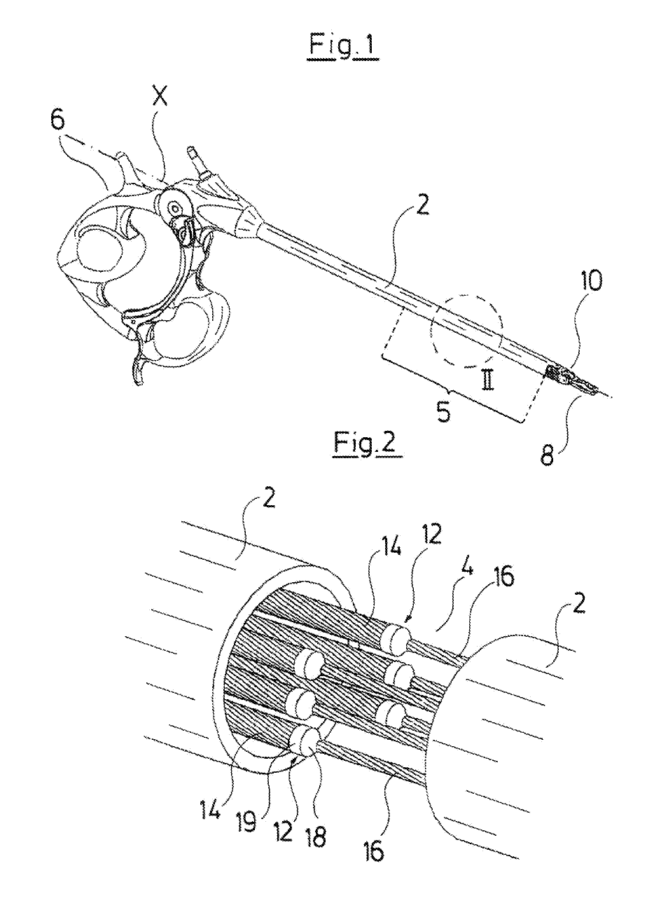

Referring to the drawings, the endoscopic instrument schematically represented in FIG. 1 and in a part view in FIG. 2 comprises an instrument shank 2 in the form of an elongated hollow body, through which several pull elements 4 pass in an axially movable manner along its longitudinal axis X. A handle 6 which is connected to the pull elements 4 is arranged on the proximal side of the instrument shank 2. A tool 8 in the form of a forceps jaws is connected to the instrument shank 2 via a bending joint 10, at the distal side which is to say distally of the instrument shank 2. The bending joint 10 thereby comprises two pivot axes transverse to the longitudinal extension of the instrument shank 2, about which axes the bending joint 10 can be bent or angled. At least one of the pull elements 4 is connected to the bending joint 10 per pivot axis, for the control of the bending. All further pull elements 4 engage through the bending joint 10 and are connected to the tool 8 in a movement-transmitting manner.

The pull elements 4 in a distal half 5 of the instrument shank 2, in front of the bending joint 10 each comprise a coupling location 12, at which a proximal first section 14 of the pull element 4 is connected to a distal second section 16 of the pull element 4. A transition piece 18 is arranged in an outer peripheral manner at the coupling location 12, on the first section 14 as well as on the second section 16, with a positive fit friction fit and/or non-positive fit, e.g. crimped. The transition piece 18 can alternatively be soldered or bonded.

The first section 14 is designed in a mechanically more stable manner compared to the second section 16, which is to say that the first section 14 has a reduced extensibility and in particular is designed with more tensile strength. The second section 16 is designed in a more flexible manner compared to the first section 14, which means to say that it has a greater flexibility than the first section 14.

The second sections 16 of the pull elements 4 pass through 10 the bending joint 10 or engage on this for its movement. The flexible second sections 16 of the pull elements 4 are thus arranged in the region of the bending and can thus follow each position of the bending joint 10. The second sections 16 form the connection to the tool 8. The pull elements in the bending joint 10 are deflected via bent or arcuate guides, in particular rollers, whose diameters are smaller than the diameter of the instrument shank 2. I.e., the pull elements are deflected over radii of curvature which are smaller than half the diameter of the instrument shank. This demands a high flexibility, in order to ensure a movability of the pull element through the bending element 10, even with the bending of this element, in order to be able to actuate the tool 8 via the pull element 4.

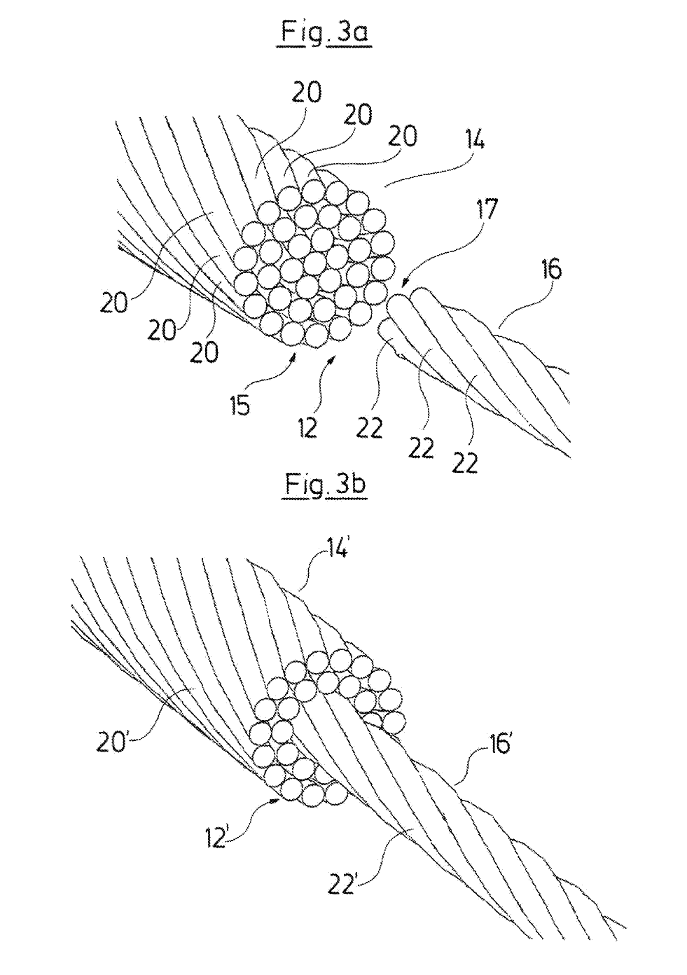

The first sections 14 as well as the second sections 16 of the pull elements 4 are formed from strands 20, 22 which are twisted with one another (FIG. 3a). The strands 20, 22 have the same diameter. Both sections 14, 16 are each designed in a pliable manner. The first section 14 thereby has a greater number of strands 20 than the second section 16 has strands 22, which is why the first section 14 has a greater cross section than the second section 16 and thus can be designed in a mechanically more stable and flexurally more rigid, in particular tensionally stronger manner. Alternatively or supplementarily, the strands 20 of the first section 14 can consist of a different material or have a different geometry, than the strands 22 of the second section 16. Accordingly, the second section 16 has a greater flexibility or pliability than the first section 14. This increased flexibility of the second section 16 compared to the first section 14 in particular allows the second section 16 to have a necessary flexibility and movability in the region of the bending joint 10.

With the first embodiment of the coupling location 12 of a pull element 4 and which is schematically represented in FIG. 3a, the first section 14 has an end 15, and the second section 16 has an end 17, wherein the ends 15, 17 face one another. The ends 15, 17 facing one another, as is shown in FIG. 2, are enclosed in an outer peripheral manner with a positive and non-positive fit by a transition piece 18, for forming a continuous pull element 4. The transition piece 18 is additionally connected to the first and second sections 14, 16 with a material fit by way of soldering for example. The transition piece 18 is designed as a truncated cone, at whose one axial truncated end extending in the direction of the first section 14 a cylindrical sleeve 19 is formed. The cylindrical sleeve 19 at the outer periphery encloses the strands 20 at the end 15 of the first section 14, and the truncated cone of the transition piece 18 encloses the ends of the strands 20 together with the end 17 of the second section 16. The transition piece 18 thus on the one hand prevents the strands 20 of the first section 14 and the strands 22 of the second section 16 from fraying. On the other hand, the two sections 14, 16 are held together at the coupling location 12 by way of the transition piece 18. The ends 15, 17 of the two sections 14 and 16 can engage into one another in an alternative design which is not represented, and this further increases the positive fit and non-positive fit at the coupling location 12.

With regard to the coupling location 12′ shown in FIG. 3b, it is the case of and alternative embodiment of the coupling location 12. An inner line of strands 20′ in the coupling location extends out of the end 15′ of the first section 14 ‘ and thereby in its further extension forms the second section 16’ of the pull element 4. The strands 22′ of the second section 16′ are thus designed as one piece with an inner part of the strands 20′ of the first section 14′. This leads to a high strength of the connection between the two sections 14′, 16′ since no connection of two loose ends is necessary. A transition piece 18 which is not represented in FIG. 3b surrounds the two sections 14′, 16′ in an outer-peripheral manner at the coupling location 12′, in an manner analogous to the previous description, secures the free ends of the strands 20′ of the first section 14′ and connects these to the inner line of the strands 20′.

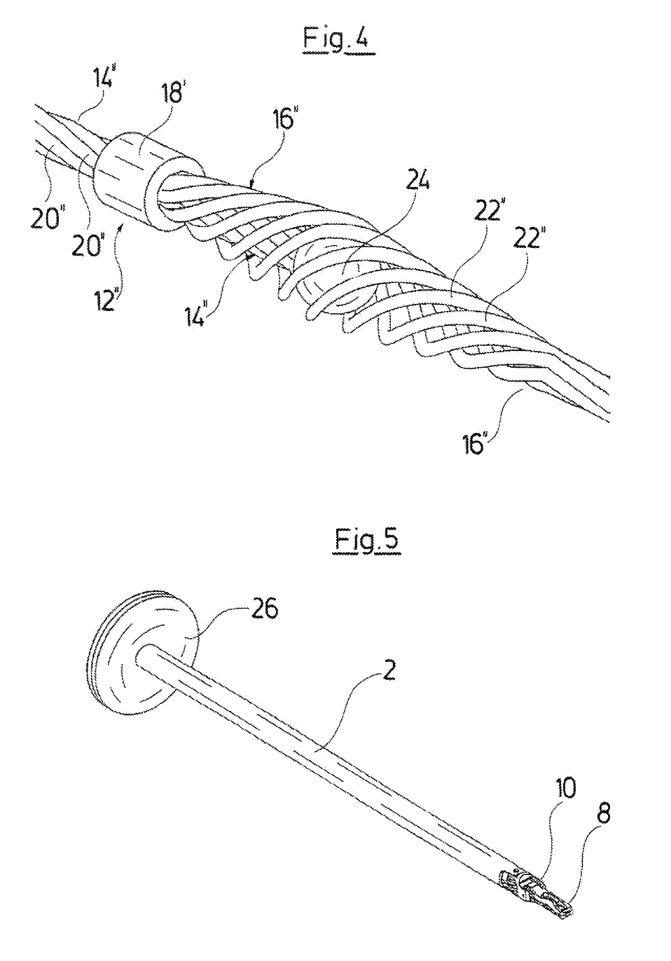

A further alternative coupling location 12″ is represented in a schematic manner in FIG. 4. A spherical thickening 24 is formed or integrally formed on the end of the first section 14″ formed from strands 20″, said end facing the second section 16″. The thickening 24 thereby can be formed by way of melting and reshaping the ends of the strands 20″, but a suitable thickening, here in the shape of a ball, can also be soldered, bonded or molded onto the end of the strands 20″. The strands 22″ of the second section 16″ embrace the thickening 24 as well as the facing end of the first section 14″ at the outer periphery. In a manner facing the first section 14″, a transition piece 18′ engaging behind the thickening 24 in the form of an annular clip is arranged at the end of the second section 16″. The transition piece 18′ is designed as a hollow cylinder piece, through which the first section 14″ engages. The end of the second section 16″ which faces the first section 14″, together with the first section 14″ is enclosed by the transition piece 18′ and thus secures the free ends of the strands 22″ of the second section 16″ on the outer periphery of the first section 14″. The transition piece 18′ can additionally be connected, for instance soldered to the first and second section 14″, 16″. The diameter of the spherical thickening 24 is larger than the inner diameter of the transition piece 18′, so that the transition piece 18′ engages behind the thickening 24 with a positive fit. Thus, a pull force transmission from the thickening 24 via the transition piece 18′ onto the second section 16″ is rendered possible.

According to the schematic representation in FIG. 5, an alternative embodiment of the endoscopic instrument has an interface 26 instead of a handle 6. The interface 26 is designed in order to connect the endoscopic instrument to a medical apparatus such as a medical robot arm. For this, the interface 26 comprises transition elements which are connected to the pull elements 4 engaging through the instrument shank 2 and which come into connection with the medical instrument in a manner such that the pull elements 4 and thus the tool and/or, inasmuch as is present, the bending joint 10, can be actuated by way of the medical apparatus.

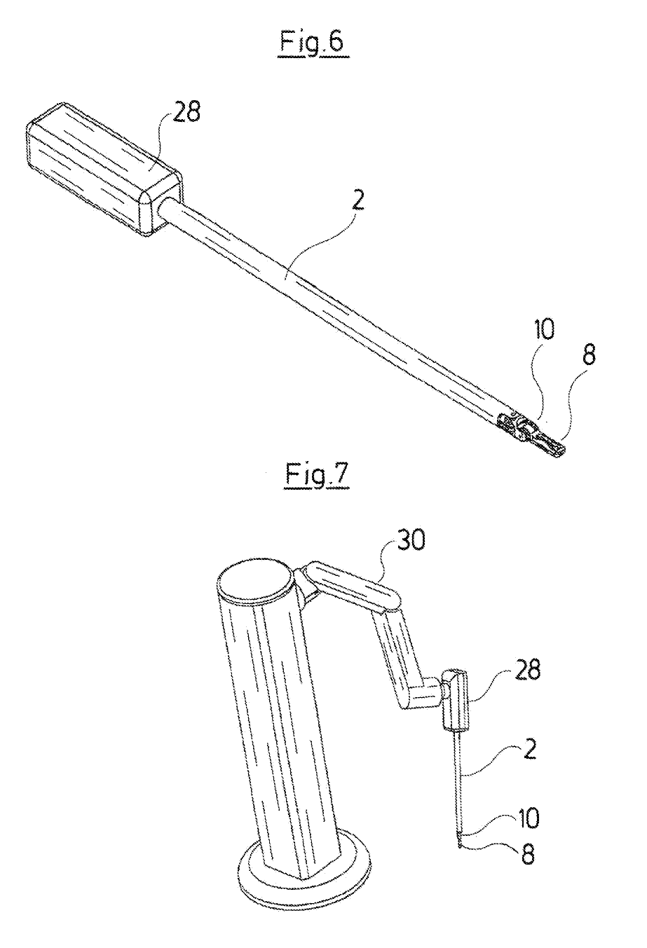

With a further alternative embodiment, the interface is formed by a drive unit 28 for actuating the pull elements (FIG. 6). The drive unit 28 thereby comprises drive elements. via which the pull elements 4 are moved and thus the tool 8 and/or the bending joint 10 are actuated. An interface, via which an energy supply of the drive elements is effected, is formed on the drive unit 28, for the activation of the drive elements.

The endoscopic instrument is connected to a robot arm 30 via a coupling formed on the drive unit 28 (FIG. 7). The robot arm 30 guides the movement of the endoscopic instrument and also controls the actuation of the tool 8 and/or the bending joint 10 via an activation of the drive elements.

While specific embodiments of the invention have been shown and described in detail to illustrate the application of the principles of the invention, it will be understood that the invention may be embodied otherwise without departing from such principles.

### APPENDIX

### List of Reference Numerals

2 instrument shank
4 pull element
5 distal half
6 handle
8 tool
10 bending joint
12, 12′, 12″ coupling location
14, 14′, 14″ first section
15 end of the first section
16, 16′, 16″ second section
17 end of the second section
18, 18′ transition piece
19 cylindrical sleeve
20, 20′, 20″ strands
22, 22′, 22″ strands
24 thickening
26 interface
28 drive unit
30 robot arm
X longitudinal axis of the hollow body

What is claimed is:

1. An endoscopic instrument comprising:
an elongate hollow body; and
at least one pull element axially movably mounted in the hollow body, the at least one pull element comprising a longitudinal extension comprising at least two flexible sections, wherein the at least two flexible sections comprise a first section and a second section wherein the first section has mechanical characteristics that are different from mechanical characteristics of the second section.

2. An endoscopic instrument according to claim 1, wherein the first section has a greater axial stiffness than the second section.

3. An endoscopic instrument according to claim 1, wherein the second section is more flexible manner than the first section.

4. An endoscopic instrument according to claim 1, wherein:
the first section is arranged at a proximal end of the hollow body; and
the second section is arranged at a distal end of the hollow body.

5. An endoscopic instrument according to claim 1, wherein at least one of the sections comprises twisted, stranded and/or braided strands or filaments.

6. An endoscopic instrument according to claim 1, wherein the first section has a greater cross section than the second section.

7. An endoscopic instrument according to claim 1, at least one of the sections has a covering at an outer periphery.

8. An endoscopic instrument according to claim 1, wherein the first section has different electrical and/or magnetic characteristics from the electrical and/or magnetic characteristics second section.

9. An endoscopic instrument according to claim 1, wherein the first section and the second section are nonpositively, positively and/or materially connected to one another.

10. An endoscopic instrument according to claim 1, wherein:
the first section has an end which faces an end of the second section; and
a coupling location connecting the first section to the second section is formed on and/or between the ends of the two sections which face one another.

11. An endoscopic instrument according to claim 10, wherein the coupling location is formed within a distal half or within a distal third of the hollow body.

12. An endoscopic instrument according to claim 10, wherein the longitudinal extension further comprises a transition piece arranged at the coupling location, wherein the transition piece peripherally embraces ends of the two sections which face one another.

13. An endoscopic instrument according to claim 12, wherein the first section comprises a local thickening which is embraced by a radially outwardly widened part of the second section.

14. An endoscopic instrument according to claim 13, wherein the end of the second section which faces the first section is radially tapered with respect to the widened part and embraces the first section behind the thickening.

15. An endoscopic instrument according to claim 10, wherein the longitudinal extension comprises at least one strand that passes through at least a portion of the first section and passes through at least a portion of the second section.

16. An endoscopic instrument according to claim 1, further comprising a tool, wherein a distal end of the pull element is connected to the tool in a movement-transmitting manner.

17. An endoscopic instrument according to claim 1, further comprising at least one of a handle, a grip piece and an interface for an attachment on a robot arm, wherein the at least one of the handle, the grip piece and the interface is arranged on a proximal end.
